(19) United States
(12) Patent Application Publication
(10) Pub. No.: US 2007/0009848 A1
(43) Pub. Date: Jan. 11, 2007

(54) Packaging system for pre-pasted orthodontic bracket

(76) Inventor: Lee H. Tuneberg, Sheboygan, WI (US)

Correspondence Address: PGM;Philip G. Meyers Law Office, Suite 300, 1009 Long Prairie Road, Flower Mound, TX, 75022, US

(21) Appl. No.: 11/453,558

(22) Filed: Jun. 15, 2006

Related U.S. Application Data
(60) Provisional application No. 60/690,658, filed on Jun. 15, 2005.

(51) Int. Cl. A61C 3/00; A61B 19/02

(52) U.S. Cl. 433/9; 206/63.5

(57) ABSTRACT

A packaged, pre-pasted orthodontic bracket includes an orthodontic bracket, a layer of light-curable adhesive is disposed on the base and an elastomeric cover for protecting the light-curable adhesive, the cover including a lip or upper edge portion that is secured the periphery of a central portion of the bracket at a location where the central portion is narrower than the base of the bracket.

This application claims priority of U.S. Provisional Ser. No. 60/690,658, filed Jun. 15, 2005.

### TECHNICAL FIELD

The invention relates to the packaging of orthodontic devices, in particular to a packaged, pre-pasted orthodontic bracket wherein the prepasted portion of the bracket is protected with an elastomeric cover.

### BACKGROUND OF THE INVENTION

Modern orthodontic techniques include repositioning teeth that are misaligned, too close together or otherwise mis-positioned. In order to reposition the teeth, the teeth are connected to an arch wire that serves as a guide, urging the teeth into the desired position and orientation. In order to connect the teeth to the arch wire, small brackets with slots for receiving the arch wire are attached to the teeth. The ends of the arch wire are normally attached to the molars with another orthodontic appliance known as a buccal tube. In some cases, the bracket or tube is brazed or welded or brazed to a metal band placed around the patient's tooth. However, the preferred method of attaching brackets to a patient's teeth is by means of an adhesive. Bonding brackets to a patient's teeth is more comfortable for the patient, cosmetically more appealing and faster in practice than the use of metal bands.

Applying an appropriate amount of adhesive to the base of an orthodontic appliance such as a bracket or buccal tube can be a time consuming and tedious task for the orthodontist or his or her assistant. Too little adhesive may result in the bracket coming loose from the tooth after installation while an excessive amount of adhesive will be pushed out from between the bracket and tooth, requiring removal. Further, while two-part chemically curing adhesive systems are available for bonding orthodontic brackets to a patient's teeth, many practitioners prefer photo sensitive adhesives that are cured upon exposure to light in the visible spectrum for a relatively short period. Consequently, time is also a factor insofar as the adhesive applied to the bracket can only be exposed to visible light for a limited period before the bracket is positioned on the tooth.

In order to address these issues, pre-pasted orthodontic appliances were developed. Pre-pasted orthodontic appliances have an adhesive, normally a photo sensitive, light curing adhesive, applied to the base of the bracket before the bracket is packaged. Pre-pasted brackets reduce the amount of time required to install brackets on the patient's teeth by eliminating the need for the orthodontist to apply the adhesive to the bracket. Pre-pasted brackets also reduce the amount of wasted adhesive as the manufacturer can precisely control the amount and placement of the adhesive on the base of the bracket.

Packaging for brackets with a pre-applied light curing adhesive must protect the adhesive from exposure to light, as well as from contamination and evaporation. Further, if the adhesive adheres to the package, the adhesive coating may be disturbed when the bracket is removed from the package. Thus, in some cases, the pre-pasted portion of the orthodontic appliance is isolated from the package material with a release coating or layer. In addition, the package should present the bracket to the orthodontist in a fashion that facilitates quick and convenient removal of the bracket from the package.

### SUMMARY OF THE INVENTION

A packaged, pre-pasted orthodontic bracket according to the invention includes an orthodontic bracket having a base, a central portion extending from the base, at least two opposed tie wings extending from the central portion and a layer of light-curable adhesive is disposed on the base. A cover for protecting the light-curable adhesive is disposed over the layer of light-curable adhesive and at least a lower portion of the base. The cover has a lip or upper edge portion that is secured about the central portion of the bracket to retain the cover on the bracket. Preferably, the cover is made of an elastomeric material that is stretched over the base. In one variation, the upper edge portion of the cover comprises an endless lip that sealingly engages a sloped upper surface of the base of the bracket around the periphery of the central portion of the bracket where the central portion is narrower than the base. The cover may engage the exposed face of the layer of light-curable adhesive or it may be slightly spaced from an exposed face of the layer of light-curable adhesive.

### BRIEF DESCRIPTION OF THE DRAWINGS

FIG. 1 is a cross sectional view of an orthodontic bracket package assembly according to the invention;

FIG. 2 is a cross sectional view of an alternate embodiment of the orthodontic bracket assembly of FIG. 1;

FIG. 3 is a top view of the orthodontic bracket package assembly of FIG. 2 with the orthodontic bracket omitted and the package in an un-stretched condition;

FIG. 4 is a cross sectional view of a second alternate orthodontic bracket package assembly according to the invention; and

FIG. 5 is a cross sectional view of a third orthodontic bracket package assembly according to the invention.

### DETAILED DESCRIPTION

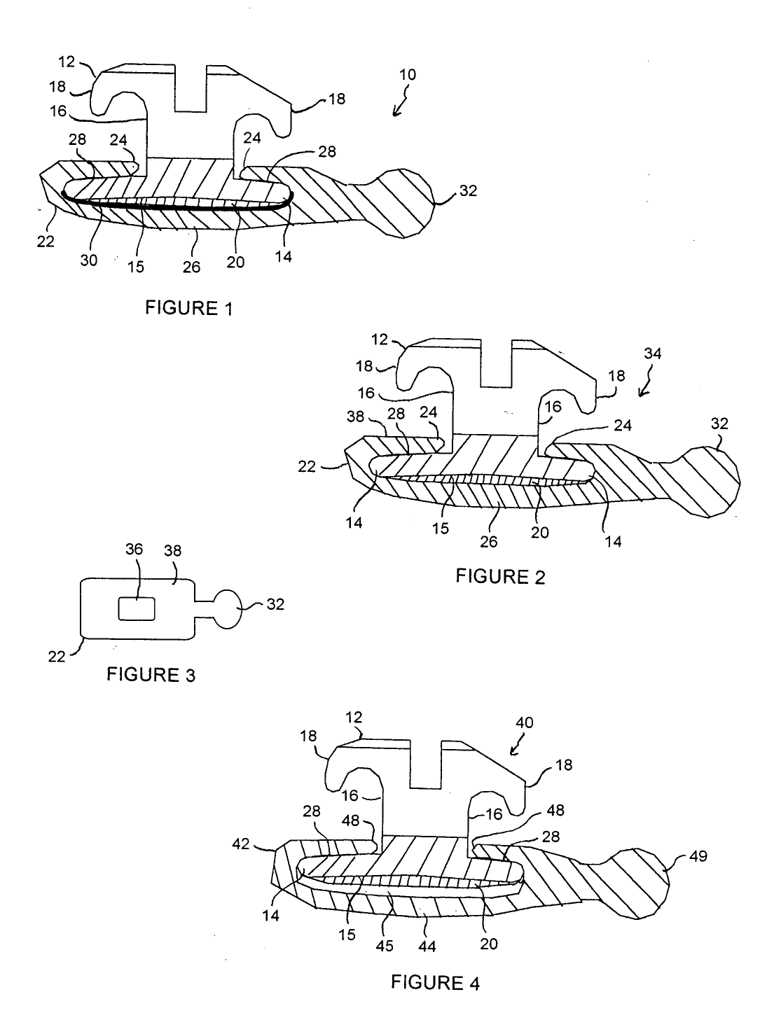

Referring to FIG. 1, an orthodontic bracket package assembly 10 includes an orthodontic bracket 12 having a base 14, a central portion 16 extending from the base and at least two opposed tie wings 18 extending from the central portion. A light curable adhesive paste layer 20, typically curable with visible light in the 400-510 nm wavelength range, is adhered to the bottom surface 15 of base 14 for adhering the bracket to a patient's tooth. As illustrated, an oval, relatively thin, elastomeric cover 22 is disposed over light curable paste 20 and base 14 to protect the paste from contamination.

Cover 22 is formed from an elastomeric material such as silicone, polyurethane, polyethylene, polypropylene or polyester, preferably with a hardness of 70 A Shore or less. Preferably, cover 22 is relatively thin, on the order of 0.015 inches in thickness. The elastomeric material may be filled with a light blocking agent or filler such as carbon black to protect the paste layer from exposure to light and premature curing. As illustrated, cover 22 is formed with an upper edge portion or endless lip 24 around the perimeter of the cover that faces inwardly toward bracket 12 when the cover is stretched over the bracket. In this manner, lip 24 encircles central portion 16 at a location where the perimeter of bracket 12 is smaller than the perimeter of base 14, enhancing retention of cover 22 on bracket 10 since lip 24 must be stretched to remove the cover from the bracket. Preferably, lip 24 engages the upper sloped surface 28 of base 14 at or adjacent the lower end of central portion 16 of the bracket 10 to provide a gas tight seal between cover 22 and bracket 10 when the cover is installed on the bracket. Cover 22 includes a tab 32 which may be grasped to stretch the cover to facilitate removal from bracket 12.

Cover 22 is provided with an opaque liner 30 positioned on the inner surface of a central portion 26 of the cover. Liner 30 serves to protect the paste layer 20 from light that would cause the paste to cure prematurely. Liner 30 may be formed from mylar, polyethylene, polypropylene or a similar material so long as the material is opaque to actinic radiation, for example in the range 400-510 nm wavelength. Liner 30 may be adhered to cover 22 with an adhesive, preferably near the center of the liner such that the liner does not inhibit stretching cover 22 when the cover is installed on bracket 12. If liner 30 protects paste layer 20 sufficiently from light, then a filler or blocking agent may be omitted from the elastomeric material, which may then be translucent or transparent. As shown, liner 30 has preferably has dimensions slightly greater than the underlying paste layer 20 to ensure complete coverage.

In an alternate embodiment, illustrated in FIGS. 2 and 3, a bracket and package assembly 34 is substantially identical to bracket and package assembly 10 of FIG. 1 with the omission of liner 30. In this variation, central portion 26 of cover 22 contacts the exposed face of paste layer 20. To prevent central portion 26 from sticking to paste layer 20 when cover 22 is removed, the inner surface of central portion may be coated with a release coating such as silicone. As illustrated in FIG. 3, cover 22 is formed with an opening 36 in an upper portion 38 of the cover through which base 14 of bracket 12 is inserted to install the cover on the bracket. Opening 36 is smaller than base 14, requiring that the opening be stretched to install the cover on bracket 12. Once installed, the tension of stretched cover 22 serves to retain the cover on the bracket and facilitate sealing of lip 24 against sloped surface 28 of bracket 12.

Turning to FIG. 4, in a second alternative embodiment, a bracket and package assembly 40 includes a thin, elastomeric cover 42 having central portion 44. Cover 42 includes a lip 48 that encircles central portion 16 of bracket 12, forming a seal against upper sloped surface 28 of the bracket. As illustrated, cover 42 is formed with sufficient material and thickness such that a small gap 45 separates central portion 44 from paste layer 20 when the cover is installed on bracket 12. In this manner, central portion 44 of cover 42 is slightly spaced from exposed face of paste layer 20, minimizing or avoiding adhesion of cover 42 to paste layer 20. Cover 42 is also provided with a tab 49 to facilitate removal of the cover from bracket 12.

Covers such as those described above could be applied manually to pre-pasted brackets, but are preferably applied by a machine that robotically engages and stretches opening 36 as it pulls the cover over the pre-pasted bracket, which is held in a fixture. To make this process easier, it is preferred according to the invention to make the inner edge of the cover thinner adjacent opening 36 than elsewhere. Lip 24 may be inwardly tapered for this purpose.

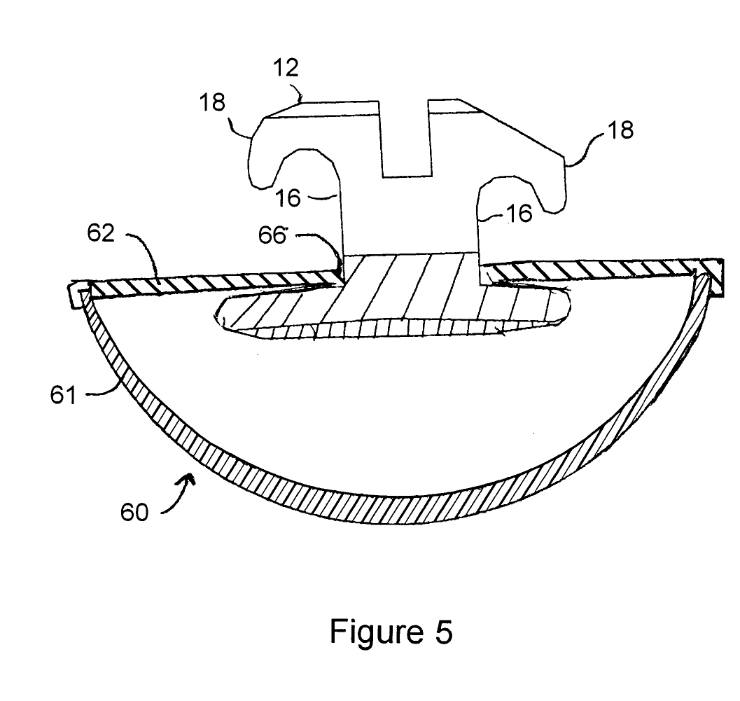

Referring to FIG. 5, a composite cover or enclosure 60 includes a concave shell or bowl 61 made of generally rigid plastic having a sheet of elastomeric material 62 secured about its rim 63. Sheet 62 has a central hole 66 similar in shape to opening 36 through which the pre-pasted bracket can be inserted to the position shown. This embodiment makes it possible to insert the lower end of the pre-pasted bracket through the opening 36 one end at a time while supporting the cover 60 by the rigid concave shell 61. The cover 60 can be removed at the time of use by grasping shell 61 and the protruding portion of prepasted bracket 12 and pulling them apart.

While certain embodiments of the invention have been illustrated for the purposes of this disclosure, numerous changes in the method and apparatus of the invention presented herein may be made by those skilled in the art, such changes being embodied within the scope and spirit of the present invention as defined in the appended claims. For purposes of the invention and the claims, use of relative terms such as “substantially”, “generally” and “about”, unless otherwise specifically defined, are meant to permit functionally insignificant variations from exact mathematical values that might otherwise be implied from the use of specific numbers. With respect to properties such as rigidity, for example, the words “generally rigid” or “substantially rigid” mean that total rigidity is not required as long as the piece does not collapse under normal use as described in the specification.

1. A packaged, pre-pasted orthodontic bracket, comprising:
an orthodontic bracket having a base, a central portion extending from the base, at least two opposed tie wings extending from the central portion, and a layer of light-curable adhesive disposed on the base; and
a cover disposed over the layer of light-curable adhesive and at least a lower portion of the base.

2. The packaged, pre-pasted orthodontic bracket of claim 1, wherein the cover has an upper edge portion that is secured about the central portion of the bracket.

3. The packaged, pre-pasted orthodontic bracket of claim 2, wherein the cover is made of an elastomeric material that is stretched over the base.

4. The packaged, pre-pasted orthodontic bracket of claim 3, wherein the upper edge portion comprises an endless lip that sealingly engages the bracket about the periphery of the central portion at a location where the central portion is narrower than the base.

5. The packaged, pre-pasted orthodontic bracket of claim 1, wherein a central portion of the cover engages an exposed face of the layer of light-curable adhesive.

6. The packaged, pre-pasted orthodontic bracket of claim 1, wherein a central portion of the cover is slightly spaced from an exposed face of the layer of light-curable adhesive.

7. The packaged, pre-pasted orthodontic bracket of claim 1, wherein the cover comprises an elastomeric material that is stretched over the base, the cover further comprising a tab extending from a side of the cover to enable the user to stretch the cover.

8. The packaged, pre-pasted orthodontic bracket of claim 1, wherein the cover comprises a substantially rigid shell having a sheet elastomeric material secured over an open side thereof.

9. A method of packaging a pre-pasted orthodontic bracket comprising:
inserting a base of the bracket into an elastomeric cover having a stretchable opening including an endless lip around the opening;
positioning the lip over the base of the bracket adjacent a central portion of the bracket extending from the base to at least two tie wings.

10. The method of claim 9 further comprising positioning the lip in sealing engagement with the base of the bracket about the periphery of the central portion at a location where the central portion is narrower than the base.

11. The method of claim 9 further comprising positioning the lip in sealing engagement with the central portion of the bracket at a location where the central portion is narrower than the base.

12. The method of claim 9 further comprising stretching the cover over the base to enclose a layer of adhesive applied to the base of the bracket.

13. The method of claim 12 wherein a central portion of the cover is positioned slightly spaced from an exposed face of the layer of a light-curable adhesive after installation of the cover on the bracket.

14. The method of claim 9 further comprising placing a placing an opaque material on the central inside surface of the cover and positioning the cover over the base with the opaque material disposed between the cover and a layer of adhesive applied to the base of the bracket.

15. The method of claim 9 further comprising placing a placing a release layer on the central inside surface of the bracket and positioning the cover over the base with the release layer disposed between the cover and a layer of adhesive applied to the base of the bracket.

16. The method of claim 9 wherein the cover comprises a sheet of elastic material secured over a substantially rigid shell, the method comprising inserting the base of the bracket through a stretchable opening in the sheet such that the base of the bracket is positioned inside the shell.

17. The method of claim 15 further comprising positioning the lip in sealing engagement with the base of the bracket about the periphery of a central portion at a location where the central portion is narrower than the base of the bracket.

18. A packaged, pre-pasted orthodontic bracket, comprising:
an orthodontic bracket having a base, a central portion extending from the base, at least two opposed tie wings extending from the central portion, and a layer of light-curable adhesive disposed on the base; and
an enclosure formed from an elastic material having a opening for receiving the base of the bracket including an endless lip around the opening for engaging the upper surface of the base of the bracket to provide a gas tight seal between the cover and the bracket.

19. The packaged, pre-pasted orthodontic bracket of claim 18 wherein the base of the bracket is positioned in the enclosure with the central portion of the bracket extending through the opening where the central portion is narrower than the base of the bracket.

20. The packaged, pre-pasted orthodontic bracket of claim 18 wherein the cover further comprises an opaque portion at an inside central portion of the cover to shield the light-curable adhesive disposed on the base from actinic radiation.
